(19) United States
(12) Patent Application Publication
(10) Pub. No.: US 2003/0021810 A1
(43) Pub. Date: Jan. 30, 2003

(54) Chlorotoxin inhibition of cell invasion, cancer metastasis, angiogenesis and tissue remodeling

(76) Inventors: Harald W. Sontheimer, Birmingham, AL (US); Craig C. Garner, Birmingham, AL (US); Jessy Deshane, Hoover, AL (US)

Correspondence Address: Benjamin Aaron Adler, ADLER & ASSOCIATES, 8011 Candle Lane, Houston, TX, 77071, US

(21) Appl. No.: 10/180,420

(22) Filed: Jun. 26, 2002

Related U.S. Application Data
(60) Provisional application No. 60/301,019, filed on Jun. 26, 2001.

(51) Int. Cl. A61K 39/02

(52) U.S. Cl. 424/236.1

(57) ABSTRACT

The present invention provides methods of treating individuals having a pathophysiological conditions that involve the activity of matrix metalloproteinase-2/pro-MMP2 system, comprising the step of: administering to said individual a pharmaceutical composition comprising a pharmaceutically effective dose of chlorotoxin and a pharmaceutically acceptable carrier.

### CROSS-REFERENCE TO RELATED APPLICATION

This non-provisional patent application claims benefit of provisional patent application U.S. Ser. No. 60/301,019, filed Jun. 26, 2001, now abandoned.

### FEDERAL FUNDING LEGEND

This invention was produced in part using funds from the Federal government under NIH grant no. R01 NS 36692. Accordingly, the Federal government has certain rights in this invention.

### BACKGROUND OF THE INVENTION

1. Field of the Invention

The present invention relates generally to the fields of cell physiology, neurology, and oncology. More specifically, the present invention relates to chlorotoxin inhibition of cell invasion, cancer metastasis, angiogenesis and tissue remodeling.

2. Description of the Related Art

Animal cells contact tightly and interact specifically with each other. They also contact a complex network of secreted proteins and carbohydrates, the extracellular matrix that fills spaces between cells. This matrix helps bind cells together and also provides a lattice through which cells can move, particularly during early stages of differentiation or in neoplasia where metastatic cancer cells invade tissues. Cells interact with molecules contained in the extracelluar matrix through specialized cell surface receptors. The composition of extracellular matrix varies by tissue type and organ, with some tissues expressing highly specialized proteins. Accordingly, receptors for these proteins are highly specialized as well.

In many tissues, the glycoproteins laminin, fibronectin, collagen or vitronectin form the major constituents of extracellular matrix. Each of these glycoproteins exists in different forms often arising from the same gene by differential splicing, but acting on different cell surface receptors.

The most well characterized receptors for the extracellular matrix are members of the integrin receptor family. These receptors share a structural similarity in that each has an U. and β chain. Different combinations of α and β chains interact with different extracellular matrix molecules. For example, fibronectin interacts with α5β1 integrin, whereas vitronectin interacts with ανβ3 intergrin.

Some extracellular proteins, collagen for example, are considered to function by adhering cells to the substrate. In contrast, other extracellular proteins such as laminin, vitronectin and fibronectin are considered to promote cell migration. In the process of cell migration, cells degrade extracellular matrix proteins. This degradation process is considered to be essential not only in the normal biological process of tissue remodeling at wound sites but also during tissue inflammation in the migration of cancer cells. To accomplish this, cells release enzymes called matrix-metalloproteinases (MMP), which is a family of structurally related, Zn-containing enzymes that have the ability to break down connective tissues.

There is currently 26 known secreted matrix-metalloproteinases, i.e., MMP-1 to MMP-26 in addition to several membrane associated matrix-metalloproteinases. The activity of these enzymes is controlled both through activation of pro-enzymes and by endogenous inhibitors such as the TIMPS (tissue inhibitors of metallo-proteinases).

Inappropriate expression, release and activity of matrix-metalloproteinases constitutes part of the pathogenic mechanism associated with a wide range of diseases. These include, for example, the destruction of cartilage and bone in rheumatoid arthritis, break down and remodeling during invasive tumor growth and tumor angiogenesis, and tissue remodeling after inflammation.

The ability of the matrix metalloproteinases to degrade various components of connective tissue makes them potential targets for controlling pathological processes. For example, the rupture of an atherosclerotic plaque is the most common event initiating coronary thrombosis. Destabilization and degradation of the extracellular matrix surrounding these plaques by matrix metalloproteinases has been proposed as a cause of plaque fissuring. The shoulders and regions of foam cell accumulation in human atherosclerotic plaques show locally increased expression of gelatinase B, stromelysin-1, and interstitial collagenase.

Inhibitors of matrix metalloproteinases will have utility in treating degenerative aortic disease associated with thinning of the medial aortic wall. Increased levels of the proteolytic activities of matrix metalloproteinases have been identified in patients with aortic aneurisms and aortic stenosis (Vine and Powell, 1991). Heart failure arises from a number of diverse etiologies, but a common characteristic is cardiac dilation, which has been identified as an independent risk factor for mortality (Lee et al., 1993). This remodeling of the failing heart appears to involve the breakdown of extracellular matrix. Matrix metalloproteinases are increased in patients with both idiopathic and ischemic heart failure (Reddy et al., 1993; Armstrong et al., 1994), and cardiac dilation precedes profound deficits in cardiac function (Sabbah et al., 1992).

The migration of vascular smooth muscle cells (VSMCs) from the tunica media to the neointima is a key event in the development and progression of many vascular diseases and a highly predictable consequence of mechanical injury to the blood vessel (Bendeck et al., 1994). Northern blotting and zymographic analyses indicated that gelatinase A (matrix metalloproteinase-2) was the principal matrix metalloproteinase expressed and excreted by these cells. After injury to the vessel, gelatinase A activity increased more than 20-fold as vascular smooth muscle cells underwent the transition from a quiescent state to a proliferating, motile phenotype (Pauly et al., 1994). Antisera capable of selectively neutralizing gelatinase A activity were able to inhibit vascular smooth muscle cell migration across basement membrane barrier.

The natural tissue inhibitor of metalloproteinase-2 (TIMP-2) showed blockage of tumor cell invasion in in vitro models (DeClerck et al., 1992). Studies of human cancers have shown that gelatinase A was activated on the invasive tumor cell surface (Strongin et al., 1993) and was retained there through interaction with a receptor-like molecule (Monsky et al., 1993). Inhibitors of matrix metalloproteinases have also shown activity in models of tumor angiogenesis (Taraboletti et al., 1995; Benelli et al., 1994).

A recent study by Madri has elucidated the role of gelatinase A in the extravasation of T cells from the blood stream during inflammation (Ramanic et al., 1994). This transmigration past the endothelial cell layer was coordinated with the induction of gelatinase A and was mediated by binding to the vascular cell adhesion molecule-1 (VCAM-1). Once the barrier was compromised, edema and inflammation were produced in the CNS. Also, leukocytic migration across the blood-brain barrier is known to be associated with the inflammatory response in EAE. Inhibition of the metalloproteinase gelatinase A would block the degradation of extracellular matrix by activated T cells that is necessary for CNS penetration.

A novel strategy to treat at least some renal diseases has been suggested by recent observations of matrix metalloproteinase behavior. A rat mesangial cell matrix metalloproteinase (MMP-2) has been cloned. This matrix metalloproteinase-2 is regulated in a tissue specific manner, and in contrast to other cellular sources such as tumor cell lines, it is induced by cytokines (Brown et al., 1990; Marti et al., 1993). While matrix metalloproteinase-2 can specifically degrade surrounding extracellular matrix, it also affects the phenotype of adjacent mesangial cells. Inhibition of matrix metalloproteinase-2 by antisense oligonucleotides or transfection techniques can induce a reversion of the proliferative phenotype of cultured mesangial cells to a quiescent or non-proliferative phenotype mimicking the natural in vitro behavior of these cells (Kitamura et al., 1994; Turck et al., 1996).

These studies provide the basis for the expectation that an effective inhibitor of gelatinase A/matrix metalloproteinase-2 would have value in the treatment of diseases involving disruption of extracellular matrix. Inhibitors of matrix metalloproteinases clearly have potential clinical applications in a host of diseases characterized by disturbance of extracellular matrix-cell interactions resulting in abnormal tissue remodeling (Vincenti et al., 1994; Grams et al., 1995).

The prior art is deficient in the lack of specific MMP-2 inhibitors with significant therapeutic potential for gliomas and other diseases. Further, the prior art is deficient in the lack of methods of treating an individual having a pathophysiological condition that involves the activity of matrix metalloproteinase-2. The present invention fulfills these prior art needs.

### SUMMARY OF THE INVENTION

Chlorotoxin (Cltx) is a small peptide isolated from scorpion venom that has been demonstrated to selectively bind to glioma cells and inhibit their invasion. The present invention demonstrates that the receptor for chlorotoxin on glioma cells is matrix-metalloproteinase-2 (MMP-2), an important matrix-degrading enzyme involved in glioma invasion. Chlorotoxin specifically and selectively interacts with matrix-metalloproteinase-2, but not with MMP-1, 3 & 9, all of which are upreglated in malignant glioma. The anti-invasive effect of chlorotoxin on glioma cells can be explained solely by its interactions with matrix-metalloproteinase-2. Chlorotoxin exerts a dual effect on MMP-2: it inhibits the enzymatic activity of matrix-metalloproteinase-2 in a dose-dependent manner (IC₅₀˜200 nM) and causes a reduction in the release and/or surface expression of mature matrix-metalloproteinase-2. These findings indicate that chlorotoxin is a specific matrix-metalloproteinase-2 inhibitor with significant therapeutic potential for gliomas and other diseases.

In one embodiment of the present invention, there is provided a method of method of treating an individual having a pathophysiological condition that involves the activity of matrix metalloproteinase-2 (MMP-2)/pro-MMP2 system, comprising the step of: administering to said individual a pharmaceutical composition comprising a pharmaceutically effective dose of chlorotoxin and a pharmaceutically acceptable carrier.

In another embodiment of the present invention, there is provided a method of inhibiting neoplastic cells or metastasis of neoplastic cells, comprising the step of: administering to said cells a pharmaceutical composition comprising a pharmaceutically effective dose of chlorotoxin and a pharmaceutically acceptable carrier.

In yet another embodiment of the present invention, there is provided a treating an autoimmune or inflammatory disorder in an individual in need of such treatment, wherein said disorder is dependent on the tissue invasion of leukocytes or other activated migrating cells, comprising the step of: administering to said individual a pharmaceutical composition comprising a pharmaceutically effective dose of chlorotoxin and a pharmaceutically acceptable carrier.

In yet another embodiment of the present invention, there is provided a treating pathophysiological condition involves the activity of matrix metalloproteinase-2/pro-MMP2 system in an individual in need of such treatment, wherein said condition is selected from the group consisting of treatment of atherosclerotic plaque rupture, aortic aneurism, heart failure, restenosis, periodontal disease, corneal ulceration, treatment of burns, decubital ulcers, wound repair, inflammation and pain, comprising the step of: administering to said individual a pharmaceutical composition comprising a pharmaceutically effective dose of chlorotoxin and a pharmaceutically acceptable carrier.

In yet another embodiment of the present invention, there is provided a treating a neurodegenerative disorder involves the activity of matrix metalloproteinase-2/pro-MMP2 system in an individual in need of such treatment, comprising the step of: administering to said individual a pharmaceutical composition comprising a pharmaceutically effective dose of chlorotoxin and a pharmaceutically acceptable carrier.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 shows the synthesis and purification of Recombinant His-Cltx.

FIG. 2 shows the dose dependent inhibition of migration of glioma cells by His-Cltx.

FIG. 3 shows the dose response curve of block of Cl flux by Pen-Cltx vs. His-Cltx.

FIG. 4 shows the affinity purification of 72 kD Cltx receptor.

FIG. 5 shows the affinity purified Cltx receptor stained with Coomassie.

FIG. 6 shows the affinity purified fraction from D54-MG cells.

FIG. 7 shows the His-Cltx directly binds to MMP-2.

FIG. 8 shows the MMP-2-Cltx receptor exhibits gelatinolytic activity.

FIG. 9 shows the other proteins that copurify with the Cltx-receptor MMP-2.

FIG. 10 shows the integrins, MT1-MMP and TIMP-2 copurifies with Cltx receptor-MMP-2.

FIG. 11 shows that chlorotoxin modulates matrix-metalloproteinase-2 enzymatic activity. FIGS. 11A and B show inhibition of matrix-metalloproteinase-2 activity by chlorotoxin. FIG. 11C shows the effects of chlorotoxin on cell surface gelatinolytic activity by in situ zymography. FIGS. 11D and E show inhibition of active and latent matrix-metalloproteinase-2 activity by chlorotoxin.

FIG. 12 shows chlorotoxin inhibits the release of mature matrix-metalloproteinase-2. FIG. 12A shows the inhibition of mature matrix-metalloproteinase-2 release by chlorotoxin. FIG. 12B shows chlorotoxin did not inhibit the release of VEGF. FIG. 12C shows the time- and dose-dependent effects of chlorotoxin. FIG. 12D shows chlorotoxin did not interact with MMP-1, MMP-3 or MMP-9.

FIG. 13 shows that chlorotoxin induces internalization of MMP-2.

FIG. 14 shows chlorotoxin inhibits Matrigel invasion of glioma cells by its interaction with MMP-2.

### DETAILED DESCRIPTION OF THE INVENTION

In accordance with the present invention there may be employed conventional molecular biology, microbiology, and recombinant DNA techniques within the skill of the art. Such techniques are explained fully in the literature. See, e.g., Maniatis, Fritsch & Sambrook, “Molecular Cloning: A Laboratory Manual (1982); “DNA Cloning: A Practical Approach,” Volumes I and II (D. N. Glover ed. 1985); “Oligonucleotide Synthesis” (M. J. Gait ed. 1984); “Nucleic Acid Hybridization” [B. D. Hames & S. J. Higgins eds. (1985)]; “Transcription and Translation” [B. D. Hames & S. J. Higgins eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Matrix-metalloproteinase-2 is a protein also known as gelatinase A, glatinase type IV or collagenase. Matrix-metalloproteinase-2 originates by enzymatic cleavage from promatrix-metalloproteinase-2, a 72 kD molecule that binds to the membrane associated MT1-MMP. Matrix-metalloproteinase-2 is expressed in a highly tissue specific manner, and is particularly upregulated in a number of cancers which include, for example, melanoma, breast carcinoma, glioma, pancreatic cancer, small lung cell carcinoma, ovarian carcinoma, colorectal cancer, urothelial cancer and the metastasis deriving from these cancers. Notably, matrix-metalloproteinase-2 is also involved in the process of neovascularization associated with cancers and aberrant tissue growth, including proliferative retinopathy where new aberrant blood vessels form in the retina and hepatic fibroproliferation, the process of cell proliferation during chronic hepatitis C. A further example of this function of matrix-metalloproteinase-2 is tissue remodeling in Type 2 diabetic proteinuria.

The present invention relates to pharmaceutical methods of treatment using chlorotoxin as an inhibitor of matrix metalloproteinase-2. The present invention identifies chlorotoxin as an inhibitor of matrix metalloproteinase-2, and thus useful as an agent for the treatment of a number of diseases. Chlorotoxin, a 36 amino acid peptide originally isolated from scorpion venom but now produced by recombinant molecular biology (or solid state peptide synthesis), is a specific ligand for matrix metalloproteinase-2, its precursor pro matrix metalloproteinase-2, and interacts with other modulatory molecules that are involved in the degradation of extracellular matrix. Specifically, chlorotoxin binds to a complex consisting of: proMMP-2, MMP-2, MT-MMP1, αν-integrin, TIMP2 and the extracellular matrix protein vitronectin. Chlorotoxin directly inhibits in a dose-dependent manner the enzymatic activity of matrix metalloproteinase-2 and proMMP-2 and that inhibition of matrix-metalloproteinase-2 via chlorotoxin inhibits tumors cell invasion. These inhibitory effects occur at a concentration range that makes chlorotoxin a viable therapeutic modality.

Accordingly, the present invention indicates that chlorotoxin should be a useful treatment for various pathologies that involve the activity of matrix-metalloproteinase-2/pro-matrix-metalloproteinase-2. These pathologies include, for example, melanoma, breast carcinoma, glioma, pancreatic cancer, small lung cell carcinoma, ovarian carcinoma, colorectal cancer, urothelial cancer and the metastasis deriving from these cancers. These pathologies also include the process of neovascularization associated with cancers and aberrant tissue growth. One notable example is proliferative retinopathy where new aberrant blood vessels form in the retina. Another example is hepatic fibroproliferation, the process of cell proliferation during chronic hepatitis C. Another example is tissue remodeling in Type 2 diabetic proteinuria.

Inhibition of matrix-metalloproteinase-2 by chlorotoxin would also treat inflammatory and chronic nervous system diseases that employ activity of matrix-metalloproteinase-2 for tissue remodeling. These include demyelinating diseases such as multiple sclerosis where matrix-metalloproteinase-2 mediates blood-brain-barrier breakdown, tissue destruction and infiltration of immune cells. Chlorotoxin should also be effective in pathological events such as matrix erosion in arthritis. Another example is periodonitis in which matrix-metalloproteinase-2 activity plays a significant role.

Thus, the present invention is directed to a method of treating an individual having a pathophysiological condition that involves the activity of matrix metalloproteinase-2/pro-matrix-metalloproteinase-2 system, comprising the step of administering to said individual a pharmaceutical composition comprising a pharmaceutically effective dose of chlorotoxin and a pharmaceutically acceptable carrier. The chlorotoxin may be either native chlorotoxin, synthetic chlorotoxin or recombinant chlorotoxin. The pharmaceutical composition comprises chlorotoxin and a pharmaceutically acceptable carrier. A person having ordinary skill in this art would readily be able to determine, without undue experimentation, the appropriate dosages and routes of administration of chlorotoxin. When used in vivo for therapy, the active composition(s) of the present invention is administered to the patient or an animal in therapeutically effective amounts, i.e., amounts that reduce matrix-metalloproteinase-2 activity and/or inhibit tumor cell invasion. Generally, the chlorotoxin is administered in a dose of from about 0.01 mg/kg of body weight of the individual to about 100 mg/kg of body weight of the individual. Chlorotoxin may be administered in a route selected from the group consisting of intravenous, intramuscular, intracranial and intrathecal administration.

In one aspect, the pathophysiological condition that involves the activity of matrix metalloproteinase-2/pro-matrix-metalloproteinase-2 system is cancer. Representative cancers which may be treated according to this method include melanoma, breast carcinoma, glioma, pancreatic cancer, small lung cell carcinoma, ovarian carcinoma, colorectal cancer and urothelial cancer.

In another aspect, the pathophysiological condition that involves the activity of matrix metalloproteinase-2/pro-matrix-metalloproteinase-2 system is metastasis of tumor cells.

In another aspect, the pathophysiological condition that involves the activity of matrix metalloproteinase-2/pro-matrix-metalloproteinase-2 system is an autoimmune or inflammatory disorders that is dependent on the tissue invasion of leukocytes or other activated migrating cells. Representative autoimmune or inflammatory disorders which may be treated according to this method include arthritis, osteoporosis, multiple sclerosis and renal disease.

In another aspect, the pathophysiological condition that involves the activity of matrix metalloproteinase-2/pro-matrix-metalloproteinase-2 system is selected from the group consisting of treatment of atherosclerotic plaque rupture, aortic aneurism, heart failure, restenosis, periodontal disease, corneal ulceration, treatment of burns, decubital ulcers, wound repair, inflammation and pain.

In another aspect, the pathophysiological condition that involves the activity of matrix metalloproteinase-2/pro-matrix-metalloproteinase-2 system is a neurodegenerative disorder. Representative neurodegenerative disorders which may be treated according to this method include stroke, head trauma, spinal cord injury, Alzheimer's disease, amyotrophic lateral sclerosis, cerebral amyloid angiopathy, AIDS, Parkinson's disease, Huntington's disease, prion diseases, myasthenia gravis and Duchenne's muscular dystrophy.

The following examples are given for the purpose of illustrating various embodiments of the invention and are not meant to limit the present invention in any fashion.

### EXAMPLE 1

Synthesis And Purification of Recombinant Chlorotoxin

As a first step towards biochemical isolation of the receptor for chlorotoxin (Cltx), a recombinant fusion protein, His-chlorotoxin was synthesized in E. coli. This molecule can be immobilized on actigel beads for the subsequent affinity purification of the chlorotoxin receptor.

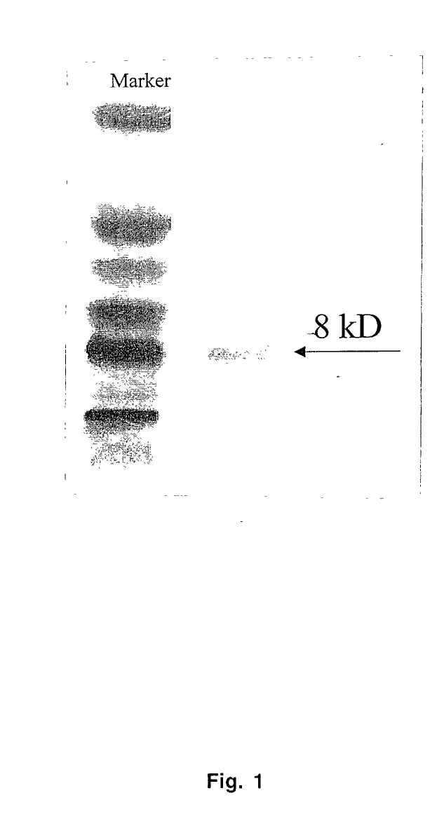

To produce His-chlorotoxin, chlorotoxin was cloned into a prokaryotic expression vector (pRsetA, Invitrogen) controlled by bacteriophage T7 promoter. This vector offers an N-terminal polyhistidine tag (6×His) which allows for purification by immobilized metal affinity chromatography (Talon Resin; CLONTECH). Briefly, BL-21 gold competent cells (Novagen, Wis.) were transformed with the plasmid DNA. An overnight culture of E. coli transformed with the expression plasmid was used to reinoculate 1 L, LB Broth and at O.D 600 nm=0.6, the culture was induced with 1 mM IPTG for 3-4 hrs and centrifuged at 3000×g for 10 min. The supernatant was removed and the pellet was resuspended in chilled extraction buffer (50 mM sodium phosphate, 300 mM NaCl, pH 7.0). The cells were sonicated on ice, 4×15 sec(level 8) with 15 sec incubation on ice between each burst. Cell lysate was sonicated again quickly following the addition of 1% Triton. The lysate was then centrifuged at 10,000×g for 20 min at 4° C. The supernatant was then transferred carefully to a new tube. Talon resin was spun at 700×g for 3 min and supernatant was removed and then resuspended in extraction buffer spun down and washed twice again. The lysate was then added to the resin and polyhistidine tagged protein allowed to bind to the beads at 4° C. for 2 hr. The resin was then washed twice with wash buffer (50 mM sodium phosphate, 300 mM NaCl, 5 mM imidazole, pH 7.0) and eluted with imidazole elution buffer (50 mM Sodium phosphate, 300 mM NaCl, 150 mM Imidazole, pH 7.0). The recombinant protein was 8 kD and was purified as a single band (FIG. 1).

### EXAMPLE 2

Biological Activity of Recombinant Chlorotoxin

In order to use the biochemically synthesized recombinant His-chlorotoxin for the isolation of the chlorotoxin receptor, the biological activity of the recombinant His-chlorotoxin was confirmed, i.e. it was demonstrated that the chlorotoxin inhibited glioma migration, inhibits Cl⁻ fluxes and paralyzes crayfish.

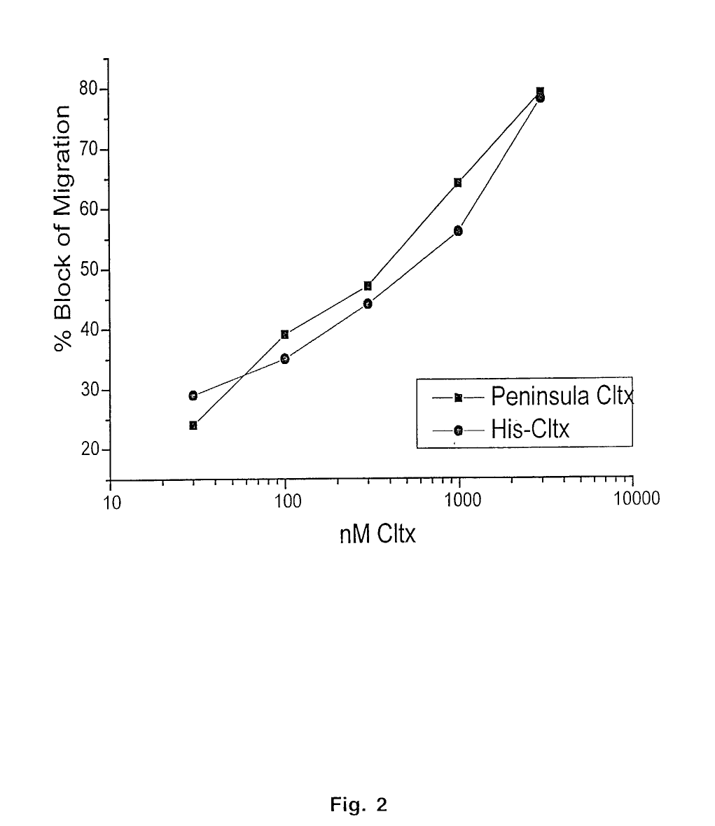

To ascertain His-chlorotoxin effects on migration, a transwell migration assay was used. Therefore, polycarbonate transwell filters (8 μm, 12 mm diameter Millipore) were evenly coated on the lower surface with vitronectin (300 μl, of 5 μg/ml vitronectin in PBS) by an overnight incubation at 37° C. The filters were then allowed to dry before plating cells. Cells at 60-70% confluency were detached using 0.53 mM EDTA, the pellet resuspended in migration buffer (serum free DMEM/F12 with 0.1% fatty acid free BSA) and plated at a density of 5×10⁴ cells/filter which fits into 24 well plate already containing migration buffer to ensure that both surfaces stay moist. 24 well plate with the filters were then incubated at 37° C. for an hour. Migration buffer was then replenished with buffer containing native peptide(Alomone) and His-Cltx at molar concentrations ranging from 30 nM, −3 μM keeping the final concentration the same in the upper and lower part of the filters and the plate was then returned for a 3 hr incubation at 37° C., 10% CO₂ humidified atmosphere. The filters coated with vitronectin alone served as positive control. Media was then aspirated from the filters and cells migrated to the lower surface of the filters were fixed in 4% paraformaldehyde for 10 min and then rinsed in PBS for 5 min. Migrated cells were stained with 1% crystal violet for 5 minutes and cotton swab was used to remove the cells from the upper chamber of the filter and 5 random fields. (1 mm²) were counted to determine the number of migrated cells and compared to untreated vitronectin control to determine the percent block of migration by His-chlorotoxin. Migration of D-54 MG glioma cells was reduced in a dose-dependent manner by His-chlorotoxin with an estimated half-maximal inhibition at 400 nM (FIG. 2).

To validate the functional efficacy of biochemically synthesized His-chlorotoxin in inhibiting Cl⁻ flux, chloride flux was measured in glioma cells in presence of His-Cltx or native peptide chlorotoxin utilizing the chloride sensitive fluorescent dye 6-methoxy-N-ethylquinolinium iodide (MEQ) as described earlier (Soroceanu et al., 1999). Briefly, 16 μM methoxy-N-ethylquinolinium was reduced by adding 12% sodium borohydride in a glass tube under constant flow of nitrogen for 30 minutes. After the reaction was completed, the organic phase which separates as a yellow oil was transferred to 1:1 mixture of ether and water and extracted twice and the organic phase was then transferred again to a new glass tube and evaporated under a constant flow of nitrogen. This was then reconstituted in serum free DMEM/F12 media to a use it at a final concentration of 5 μM. Glioma cells (D54-MG) were plated in a 96 well plate at a density of 5000 cells/well. After 24 hr from plating, cells were loaded with the dye in the dark, at 37° C. for 10 minutes. The reduced form of methoxy-N-ethylquinolinium is membrane permeable; once loaded, diH-methoxy-N-ethylquinolinium is converted to the oxidized form (MEQ), which is retained within the cells. Cells were then rinsed quickly with serum free DMEM/F12 media and then replenished again and incubated at 37° C. for an additional 10 minutes for uniform distribution of the dye into the cytoplasm of the cells.

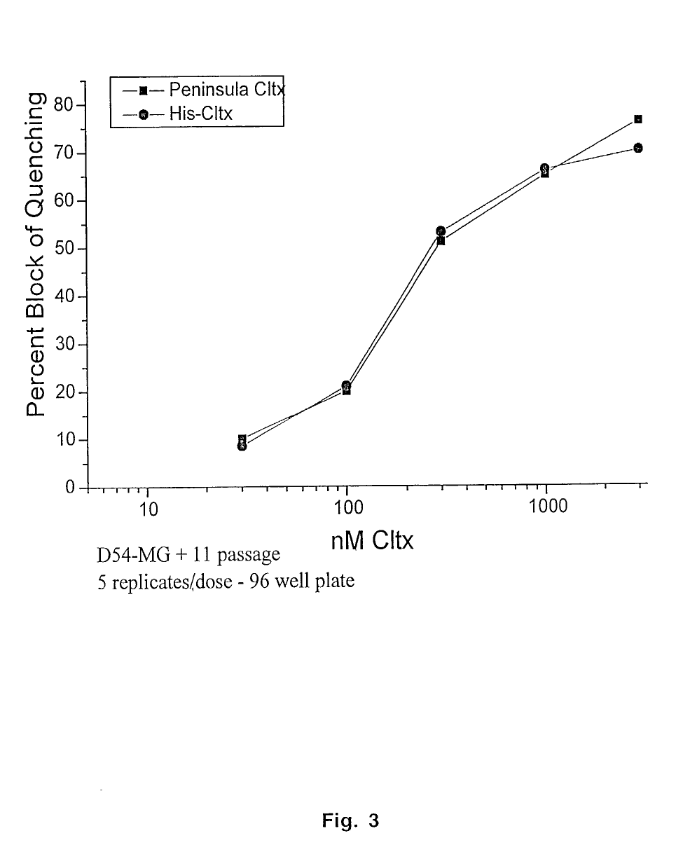

To obtain quantitative information regarding the effects of chlorotoxin on Cl⁻ fluxes, a Fluostar 403 fluorescent plate reader was used (BMG Lab Technologies, Durham, N.C.). Methoxy-N-ethylquinolinium fluorescence measurements were obtained using 355 nm excitation and 460 nm emission filters. During the recordings, cells were perfused with with solution containing 130 mM Na gluconate, 5.4 mM K gluconate, 0.8 mM MgSO₄, 1.2 mM Ca gluconate, 1 mM NaH₂PO₄, 5.5 mM glucose and the pH was adjusted to 7.4 with Tris. To obtain a hypotonic solution, the sodium gluconate was reduced to 60 mM. Replacement of chloride salts with gluconate was necessary to maintain a maximum initial fluorescence of methoxy-N-ethylquinolinium, which is quenched by collision with halide ions (Cl⁻, Br⁻, I⁻, SCN⁻). Chlorotoxin was added in hypotonic solution in concentrations ranging from 30 nM−3 μM at room temperature and multiple readings of the same microplate over a duration of 40 minutes was obtained. Data were analyzed using Fluostar software that is integrated with Excel 5.0 and percent block of quenching was calculated. Dose dependent inhibition of Cl⁻ flux was observed with an approximate half maximal inhibition at 300 nM. This was comparable to that obtained with native peptide (FIG. 3).

Crayfish paralysis Assay: Procambarus clarkii (from Atchafalaya Biological, La.) were weighed (approximately 4-7 gram, 2-3 inch optimal). Previously frozen His-chlorotoxin was thawed and pipetted into a 1.5 ml eppendorf tube to deliver 1 mg/gram crayfish. His-chlorotoxin or native peptide was injected into crayfish using a 1 ml tuberculin syringe, with ½_ inch needle (B-D # 32229424). The crayfish was turned over to expose its ventral side. The syringe was held at 90° perpendicular to the crayfish and inserted (½-¾ of the needle length), to gain access to the sub-esophageal ganglion that requires subtle, and patient manipulation through the chelicerae. The crayfish was kept out of the water and allowed to move around until paralysis occurs (30-120 seconds). They were scored based on the stiffness in the legs to determine the extent of paralysis. His-chlorotoxin when injected into the ganglion of crayfish induced sufficient paralysis, although not as efficient and sustained paralysis as observed with native peptide. Crayfish were returned to deionized water to recover.

### EXAMPLE 3

Affinity Purification of Cltx Receptor

Recombinant chlorotoxin (His-Cltx) was chemically conjugated to Actigel-ALD (Sterogene, Calif.) and then used for affinity purification of the receptor for chlorotoxin. Briefly, Actigel-ALD beads were rinsed once with 0.1% BSA in PBS (pH 7.4) and then washed three times with PBS. His-chlorotoxin was then added to Actigel-ALD (0.5 mg/ml of resin) followed by ALD-coupling solution (1M NaCNBH3) to a final concentration of 0.1 M (0.2 ml/ml resin). The suspension was agitated gently for 2 hr at room temperature or overnight incubation at 4° C. The beads were then centrifuged at 500×g in a clinical centrifuge, washed twice with PBS plus 0.1% NP-40, twice with PBS plus 0.01% Tween 20 and three times in PBS. Recombinant chlorotoxin-conjugated beads were stored in 10% glycerol and 0.02% sodium azide containing PBS to form 1:1 slurry. Cultured glioma cells were washed twice with cold PBS, scraped with cell scrapers and pelleted at 2000×g for 5 min at 4° C. The cell homogenates were prepared by resuspending cell pellet in 1.0 ml homogenization buffer (10 mM Tris ⁻Cl (pH 7.5), 0.32 M sucrose, 1 mM MgCl₂, 5 mM CaCl₂ supplemented with 10 μ/ml of protease inhibitor cocktails I and II (cocktail I: 1 mg/ml leupeptin, 1 mg/ml antipain, 5 mg/ml aprotinin, 10 mg/ml benzamidine hydrochloride, 10 mg/ml soybean trypsin inhibitor and cocktail-II: 1 mg/ml pepstatin, 30 mM phenylmethanesulfonyl fluoride in dimethyl sulfoxide) and homogenizing in glass tissue grinders for 1 minute with incubations on ice at 1 minute intervals. Cell debris was spun down at 2000×g for 5 min at 4° C. and the supernatant was collected and centrifuged at 100,000×g in Beckman Instruments T70.1 rotor for 60 min at 4° C. Pellet which represents the total cell membrane fraction was resuspended in homogenization buffer (supplemented with protease inhibitors) and containing 1% SDS and 7 fold excess volume of 1% Triton X-100 and further heated to 48° C. for 5 minutes. This lysate was then precleared with unconjugated Actigel-ALD beads for 4 hr at 4° C. The beads were spun down and the supernatent removed and incubated with the His-chlorotoxin-conjugated Actigel-ALD beads for 4 hours at 4° C. or overnight. The beads were then extensively washed with the buffer before elution of the bound proteins by boiling with Laemmli SDS-sample buffer (62.5 mM Tris-HCl. pH 6.8, 10% glycerol, 2% SDS, 0.1% bromophenol blue and 600 mM β-mercapto-ethanol) for 5 minutes and the eluted proteins were separated on denaturing 8, 10 or 4-15% gradient gel by SDS− PAGE.

The receptor of chlorotoxin was identified by proteins which would directly interact with His-chlorotoxin in an overlay assay. For overlay assays, briefly, proteins processed from membrane fractions, cytosolic fractions or total cell lysates as described above were separated on 8, 10 or 4-15% polyacrylamide gel SDS-PAGE and transferred to polyvinylidene fluoride membranes. The blots were then blocked in blocking buffer (BB) consisting of 5% non fat milk, 0.1% Tween 20 in TBS for 30 minutes at room temperature and incubated with 500 nM 6× His-chlorotoxin diluted in blocking buffer for an hour at room temperature. Following 3×10 min washes in 0.1% Tween 20 plus TBS (TBS-T) the membranes were reblocked in blocking buffer for 10 minutes and then probed with monoclonal antibody against 6×His (Clonetech, 1:5000) diluted iln blocking buffer for an hour at room temperature. Subsequently, the blots were rinsed twice in TBS-T for 10 min each, reblocked in blocking buffer for 10 min at room temperature and incubated with horseradish peroxidase conjugated anti-mouse (1:1000) or alkaline phosphatase conjugated anti-mouse IgG (H+L) (1:1000; Vector Labs, Burlingame, Calif.). Blots were washed several times and developed using Enhanced Chemiluminescence Plus (ECL+Plus; Amersham) on Hyperfilm (Amersham).

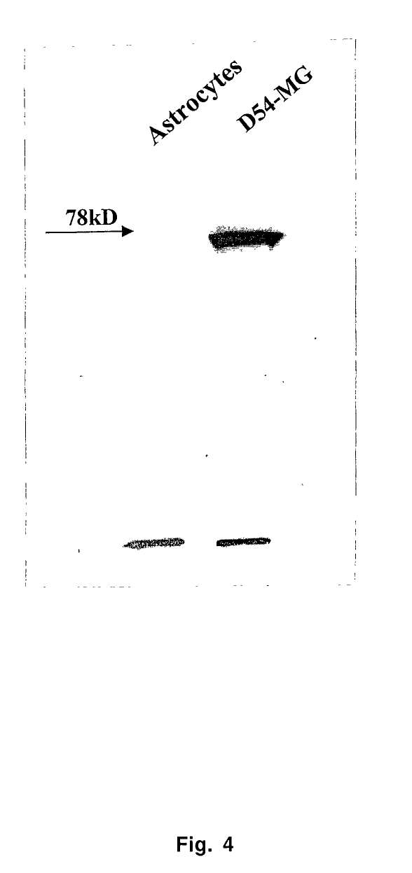

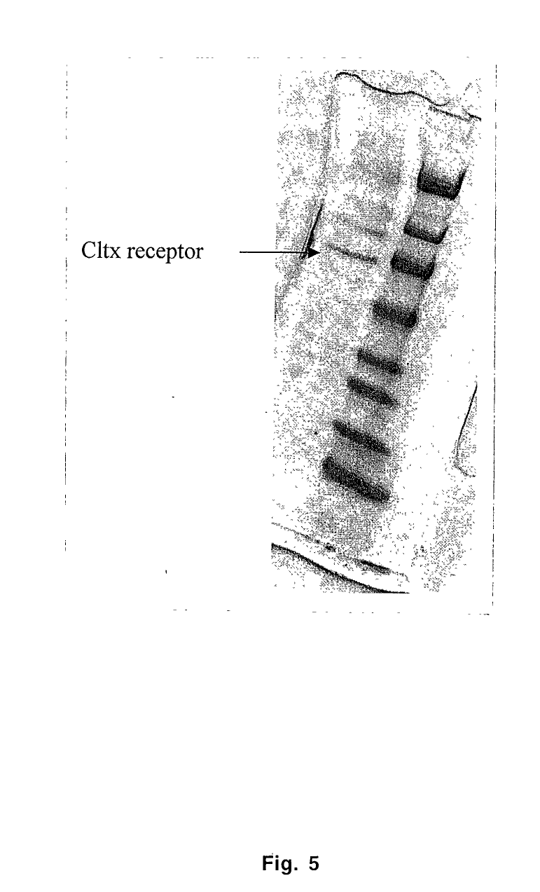

A 72 kD band was observed consistently in the overlays with His-Cltx following affinity purification with an Actigel-ALD column (FIG. 4). The identity of the receptor was determined following electrophoresis of the affinity purified fraction on a 4-15% gradient polyacrylamide gel (FIG. 5), staining with Bio-Safe Coomassie (Bio-Rad, Calif.) and excising the band of interest. The protein was then destained and trypsinized and the protein digest extract was analyzed by a MALDI-TOF mass spectrometer (PEBiosystems, Framingham, Mass.). The peptide masses were entered into MASCOT to identify the protein by searching the NCBI database. Sequence information was obtained with a Micromass Q-TOF-2 mass spectrometer (Data 1).

### EXAMPLE 4

The Cltx-Receptor is MMP-2

Following mass spectrometry and sequencing, the identity of the receptor was further confirmed by western blot analysis using a polyclonal anti-matrix-metalloproteinase-2 antibody (Sigma). Proteins were separated on a 7.5% polyacrylamide gel by SDS-PAGE. Gels were transferred onto polyvinylidene fluoride (PVDF) membranes (Immobilon-P; Millipore, Bedford, Mass.) at 200 mA for 90 minutes at room temperature and then blocked for 1 hr at room temperature in Blocking Buffer (BB) containing: 5% nonfat milk, 2% Bovine Serum Albumin (BSA), and 2% normal goat serum in Tris-Buffered Saline (TBS) plus 0.1% Tween20 (TBS-T). This step was followed by an incubation with the primary antibody diluted 1:1000 in buffer containing: 1% nonfat milk, 1% Bovine Serum Albumin and 1% Normal Goat Serum, for 2 hours at room temperature, then rinsed six times for 5 minutes each in in Tris-Buffered Saline (TBS) plus 0.1% Tween20 and reblocked for 30 minutes in blocking buffer at room temperature. Subsequently, blots were incubated with HRP-conjugated secondary antibody for 1 hour at room temperature, rinsed 6 times for 5 minutes in Tris-Buffered Saline (TBS) plus 0.1% Tween20 and developed using Enhanced Chemiluminescence Plus (ECL+Plus; Amersham) on Hyperfilm (Amersham). Western Blot analysis with an anti-matrix-metalloproteinase-2 antibody also identified a significant band of molecular weight 72 kD.

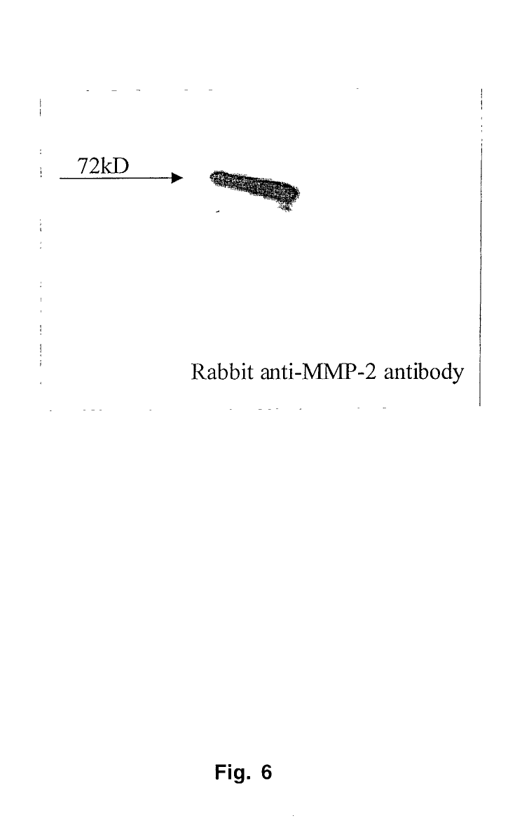

To confirm the specificity of the antibody, western blot analysis was performed as described above including other matrix metalloproteinases such as recombinant matrix metalloproteinase-1, matrix metalloproteinase-3 and matrix metalloproteinase-9 (Sigma). Immunoreactivity with the anti-matrix metalloproteinase-2 antibody was observed only in the affinity purified fraction (FIG. 6).

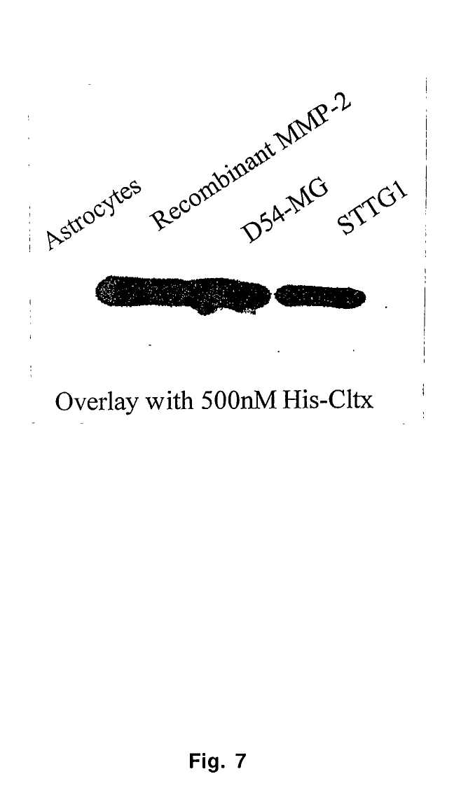

An overlay assay was also utilized to ascertain direct interaction of matrix metalloproteinase-2 and His-chlorotoxin. Recombinant purified human matrix metalloproteinase-2 was electrophoresed on a 10% polyacrylamide gel and overlay assay was performed as described earlier with 500 nM His-chlorotoxin. Significant protein bands of apparent molecular weight 72 kD which was comparable to the band detected with the recombinant matrix metalloproteinase-2 and an additional lower band possibly the active form of matrix metalloproteinase-2 was detected in the affinity purified fraction (FIG. 7).

### EXAMPLE 5

MMP-2—The Identified Cltx Receptor Exhibits Gelatinolytic Activity

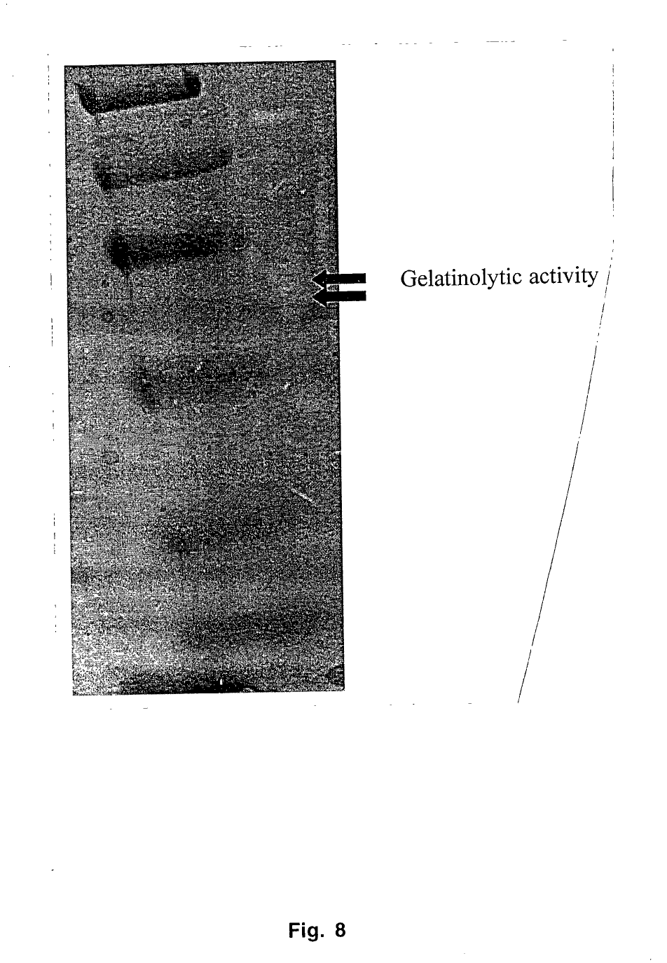

To determine the gelatinolytic activity of affinity purified receptor, matrix metalloproteinase-2, gelatin zymography was performed using 10% polyacrylamide gels containing 0.1% gelatin. Briefly, the eluate from the affinity purification column was separated by SDS-PAGE on the gel and following electrophoresis, the gel was washed with 2.5% Triton X-100 for 1 hour to remove SDS and incubated at 37° C. for 24 hr in a buffer containing 50 mM Tris-Cl, pH 8.0; 5.0 mM CaCl₂ and 1 μM ZnCl₂. The gel was then stained with Coomassie Brilliant Blue and destained quickly to reveal gelatinolytic activity as opaque unstained bands (FIG. 8).

### EXAMPLE 6

Integrins, MT1-MMP, TIMP-2 and Vitronectin Co-Purify With MMP-2

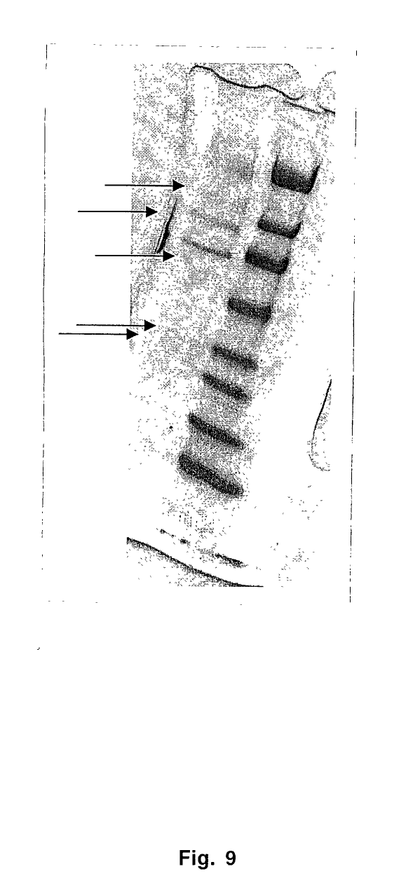

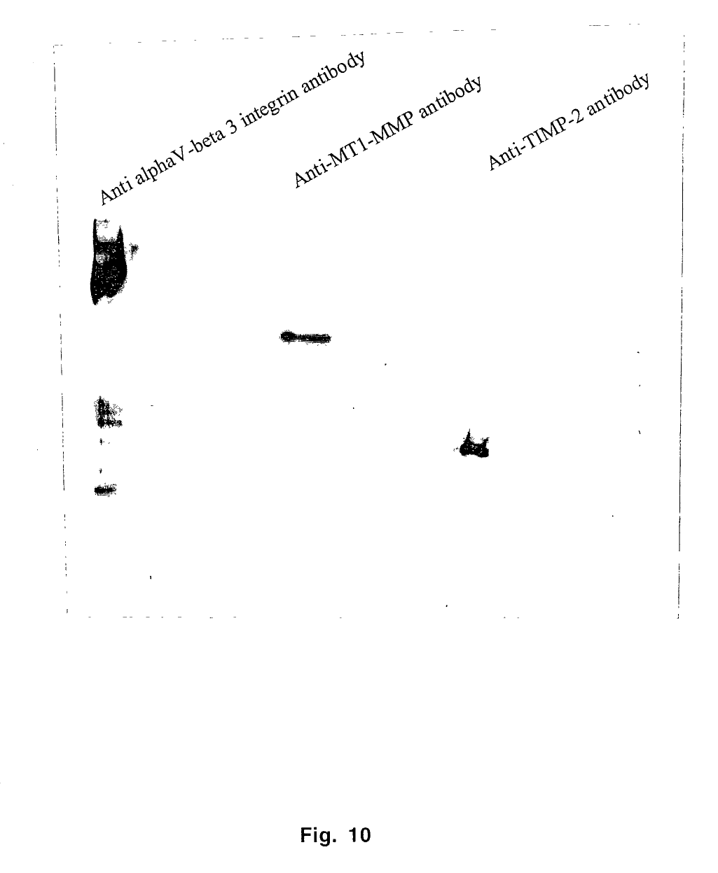

Although assays to identify proteins directly interacting with His-Cltx revealed one prominent protein, namely matrix metalloproteinase-2, other proteins co-purified with this receptor. The identity of these proteins were determined following electrophoresis of the affinity purified fraction on a 4-15% gradient polyacrylamide gel (FIG. 9), staining with Bio-Safe Coomassie (Bio-Rad, Calif.) and excising the prominent bands. The proteins were processed as described earlier. Mass Spectrometry analysis identified proteins with highly significant homology to integrin αV, MT1-MMP, TIMP-2 and Vitonectin (See Data 2). The identity of these proteins was further confirmed by western blot analysis as described earlier, utilizing specific antibodies including mouse anti-human integrin αVβP3 monoclonal antibody (Chemicon), rabbit anti-TIMP-2 (Chemicon) or rabbit anti-MT1-matrix metalloproteinase antibody (Chemicon). Protein bands of apparent molecular weights comparable to the above mentioned proteins were observed in the affinity purified fraction (FIG. 10).

### EXAMPLE 7

Chlorotoxin Modulates MMP-2 Activity

Tumor invasion, metastasis and angiogenesis require controlled degradation of extracellular matrix. Increased expression of matrix-metalloproteinases has been associated with these processes in malignant tumors of different histogenetic origin (Kahari and Saarialho-Kere, 1999). In gliomas, upregulation of matrix-metalloproteinase-2, matrix-metalloproteinase-9 and MTI-matrix-metalloproteinase characterize high grade gliomas (glioblastoma multiformae) as opposed to low grade gliomas or to non-transformed control brain tissues (Ellerbroek and Stack, 1999; Friedberg et al., 1998; Sawaya et al., 1996). Moreover, matrix-metalloproteinase-2 activity also modulates glioma cell migration and contributes significantly to their invasive potential (Deryugina et al., 1997). Consequently, several matrix-metalloproteinase inhibitors including 1-10 phenanthroline, cyclic peptides and hydroxamate derivatives have been found to effectively block migration and invasion of tumor cells (Hidalgo et al., 2001).

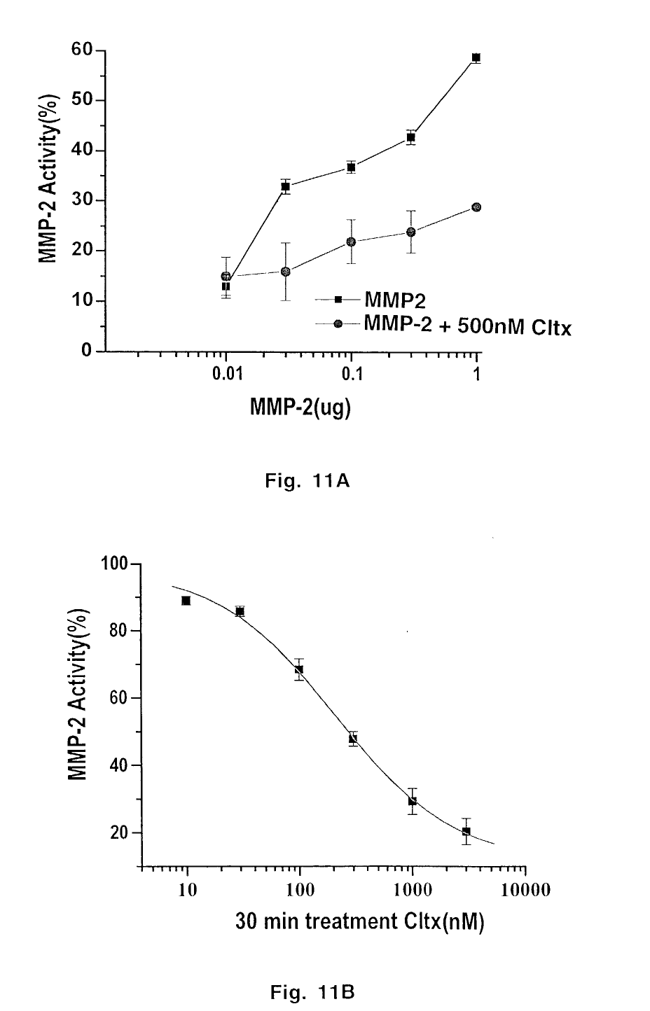

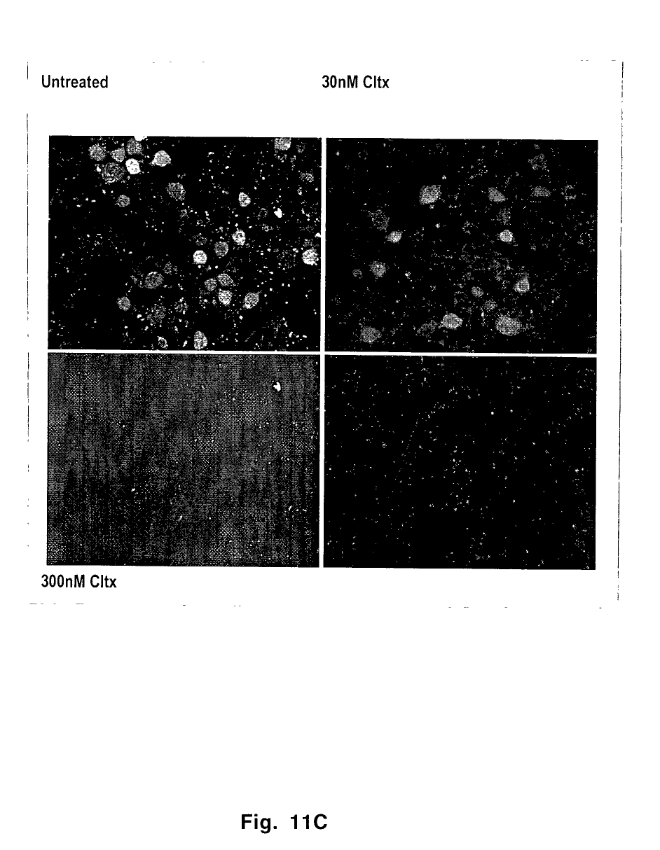

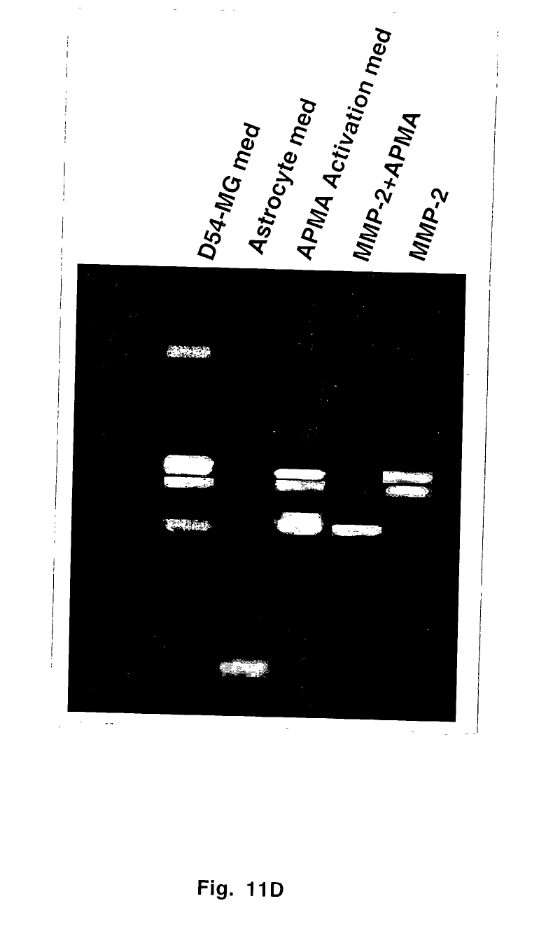

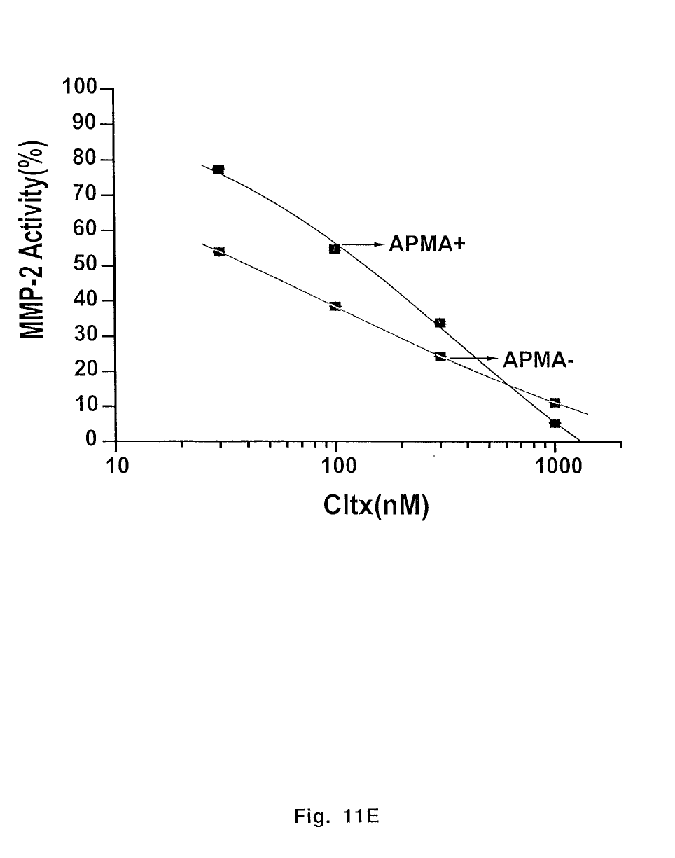

Whether the anti-migratory effect of chlorotoxin is through possible modulation of enzymatic activity of matrix metalloproteinase-2 was investigated. A matrix metalloproteinase Gelatinase activity assay (Chemicon) was utilized with recombinant human matrix-metalloproteinase-2 used as a positive control. The assay utilizes a biotinylated gelatinase substrate which is cleaved by active matrix metalloproteinase-2 and shortens the biotinylated gelatin molecules. The mixture is then transferred to a biotin-binding 96 well plate which captures the biotinylated gelatin and free biotin detected with streptavidin-enzyme complex. Addition of enzyme substrate yields a colored product which is then detected by its optical density at 450 nm. In the presence of 500 nM chlorotoxin, which is the reported IC₅₀ of chlorotxin on glioma cell invasion (Soroceanu et al., 1999), the enzymatic activity of matrix-metalloproteinase-2 was greatly reduced over a 2 log range of matrix-metalloproteinase-2 concentrations (FIG. 11A). A dose-response curve for chlorotoxin was established by determining the relative inhibition of matrix-metalloproteinase-2 activity by increasing concentrations of chlorotoxin (FIG. 11B). Chlorotoxin inhibited matrix-metalloproteinase-2 with an apparent IC₅₀ of 200 nM following 30 min treatment with chlorotoxin.

The effect of chlorotoxin on cell surface gelatinolytic activity was then investigated by in situ zymography. FITC-labeled DQ gelatin which is intramolecularly quenched (Molecular Probes, Eugene, Oreg.) was used as a substrate for degradation by gelatinases as reported earlier (Oh et al., 1999). Proteolysis by gelatinases yields cleaved FITC-gelatin peptides and the localization of this fluorescence indicates the sites of net gelatinolytic activity. Briefly, glioma cells were plated on 12 mm coverslips. After 24 hour incubation, cells were treated with 30 nM chlorotoxin, 300 nM chlorotoxin or 50 μM 1-10 phenanthroline for 30 minutes at 37° C. Untreated cells served as negative control for this experiment. Cells were then washed with PBS and then incubated with zymography reaction buffer (0.05 M Tris-HCl, 0.15 M NaCl, 5 mM CaCl₂ and 0.2 mM NaN3, pH 7.6- the high concentration of azide will prevent the gelatin from phagocytosing and thus allow cell surface gelatinolytic activity to occur) containing 100 μg/ml DQ gelatin at 37° C. overnight. At the end of the incubation period, without fixation or further washes, gelatinolytic activity of the MMP-s was localized and photographed by fluorescence microscopy and the images were acquired by Spot digital camera. Untreated glioma cells exhibited significant cell surface gelatinolytic activity.

A significant decrease in surface gelatinolytic activity was observed following treatment with 30 nM chlorotoxin, with complete inhibition in the presence of 300 nM chlorotoxin (FIG. 11C). The inhibition by 300 nM chlorotoxin was comparable to that achieved with 50 μM 1-10 phenanthroline, a well established matrix-metalloproteinase-2 inhibitor.

Tumor cells constitutively secrete a latent form of matrix-metalloproteinase-2. This latent form (˜72 kDa) is also associated with the plasma membrane and was the primary form purified in the affinity purification studies disclosed above. Secretion of active matrix-metalloproteinase-2 is a regulated complex mechanism. The latent form is converted to an activated intermediate which is then autocatalytically modified to a mature form with an apparent molecular weight of 62 kDa. Although the latent form is active and can be inhibited by chlorotoxin (FIGS. 11A, B), it is of interest to assess whether chlorotoxin could also bind and regulate the activity of the mature forms of matrix-metalloproteinase-2. To this end, D54-MG cells were treated with 1 mM APMA (aminophenylmercuric acetate), a drug that activates matrix-metalloproteinase-2 to its mature form. This treatment also allowed us to assay the release of mature matrix-metalloproteinase-2 into culture medium. Samples of conditioned serum-free medium from these cells as well as untreated D54-MG cells and cortical astrocytes were allowed to bind directly to His-chlorotoxin coated on a 96-well plate. Gelatin zymographic analysis of proteins bound to His-chlorotoxin, eluted and separated using gels demonstrated that D54-MG cells secrete all three forms of matrix-metalloproteinase-2.

The proportion of the mature 62 kDa form is increased after aminophenylmercuric acetate treatment (FIG. 11D). Although these matrix-metalloproteinase-2 isoforms were not detected in cortical astrocytes, a protein (˜20 kDa) exhibiting gelatinolytic activity that bound chlorotoxin was detected. The identity of this protein is currently unknown. A direct comparison of the inhibitory effect of chlorotoxin on the enzymatic activity of mature and latent matrix-metalloproteinase-2 is demonstrated in FIG. 11E. Chlorotoxin inhibited both enzymes in a dose-dependent fashion, but the inhibition of mature matrix-metalloproteinase-2 (after aminophenylmercuric acetate treatment) was enhanced.

### EXAMPLE 8

Chlorotoxin Inhibits the Release of Mature MMP-2

It has been demonstrated that the interaction between MT1-MMP and ανβ3 integrin promotes the activation of matrix-metalloproteinase-2. Specifically, these proteins affect the initial activation, the transient docking of the activation intermediate and the release of mature active matrix-metalloproteinase-2 at discrete regions of the cells. Since these proteins can all exist in a complex with chlorotoxin, the possibility that chlorotoxin may inhibit the release of mature matrix-metalloproteinase-2 was investigated.

For these experiments, glioma cells were plated in 96 well (5000 cells/well) or 24 well plates (2.5×10⁴/well) in serum containing medium (SCM). After overnight incubation, cell cultures were washed and incubated with serum free medium (SFM) for 24 hrs. Cells were then treated with His-Cltx at concentrations ranging from 30 nM to 3000 nM for 10 min or 30 min at 37° C. Cells were washed and replenished with serum free medium and at post-incubation periods of 10 min, 30 min and 24 hrs, conditioned media was collected from all samples and analyzed for matrix-metalloproteinase-2 activity by gelatinase activity assay as well as Western blots analysis for detection of matrix-metalloproteinase-2 protein. Cells treated with irrelevant His-protein were used as a negative control (untreated cells). Conditioned media from cells treated with 1-10 phenanthroline (50 μM), a known metalloproteinase inhibitor, under the same conditions served as positive controls for inhibition of matrix-metalloproteinase-2 enzymatic activity. Conditioned media from 10 wells were pooled for gelatinase activity assay as well as western blot analysis and data shown herein represents an average of three experiments.

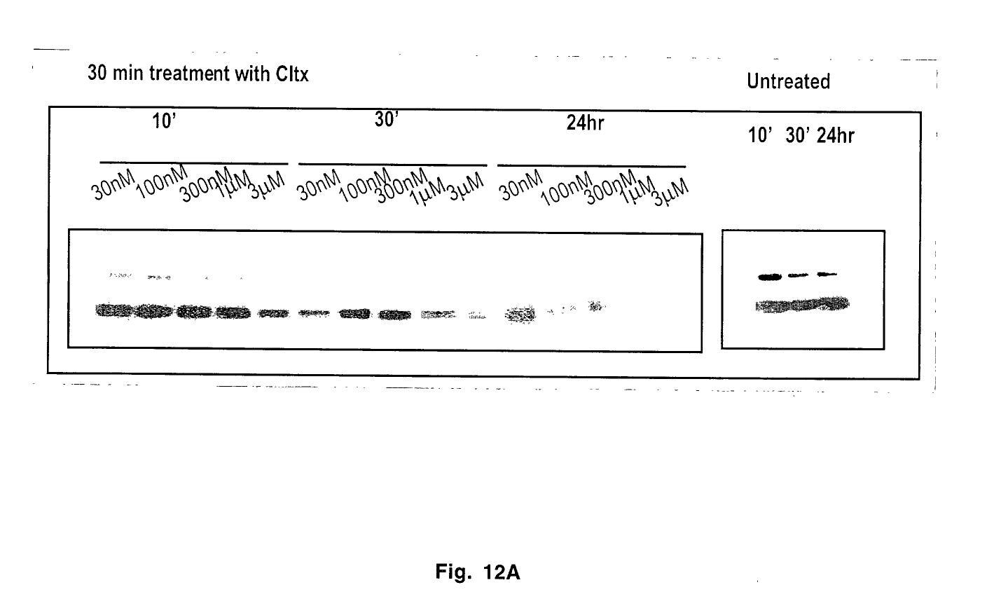

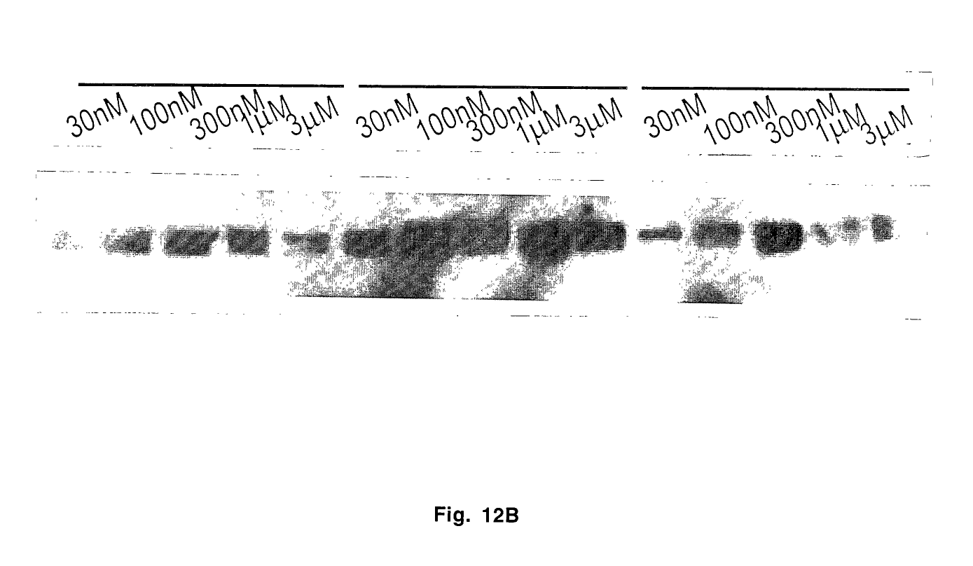

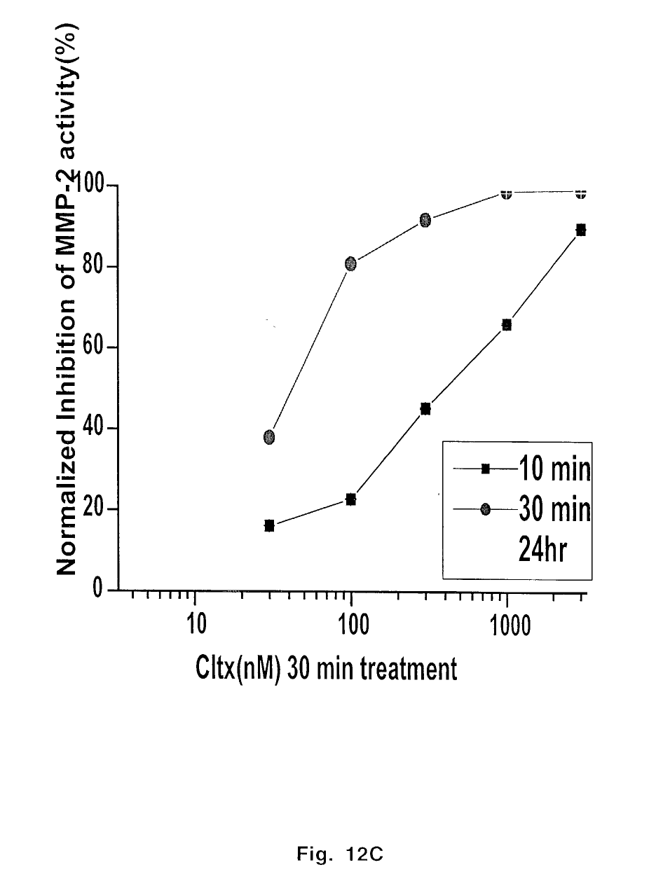

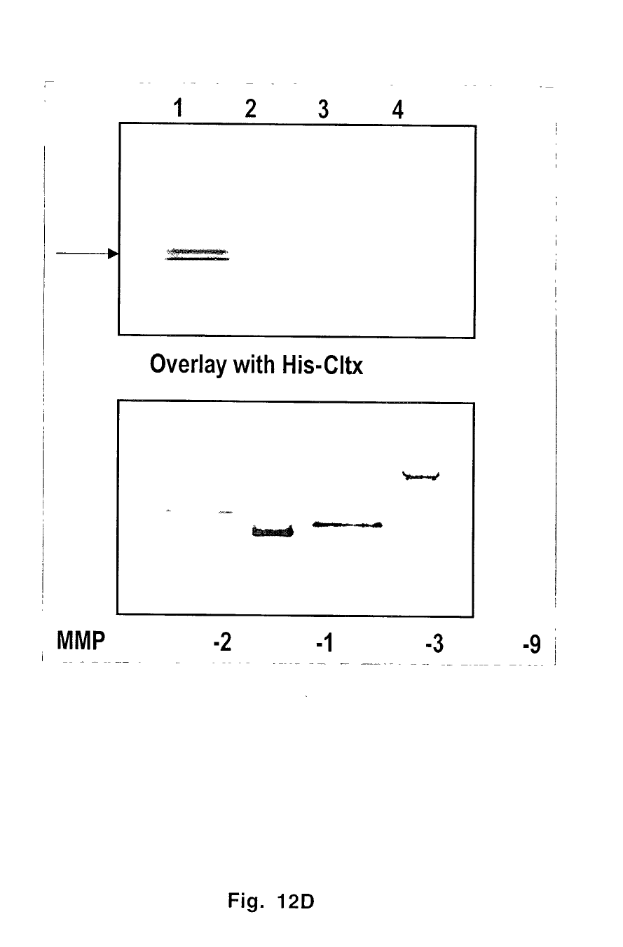

These experiments showed a significant reduction in the release of all active species of matrix-metalloproteinase-2 (FIG. 12A). This inhibition was readily detected following either a 10 or 30 min treatment, times that correlate well with the previously reported time frame for internalization of chlorotoxin into glioma cells. Lower molecular mass species indicative of degradation of matrix-metalloproteinase-2 were not detected in the conditioned media. As expected, cells treated with 1-10 phenanthroline at 50 μM, a well established inhibitor of matrix-metalloproteinase-2, also blocked the release of matrix-metalloproteinase-2 into the medium (data not shown).

To assure that these effects were specific for the release of matrix-metalloproteinase-2, these media samples were also analyzed for the release of VEGF (vascular endothelial growth factor) under identical conditions (FIG. 12B). Treatment with chlorotoxin did not affect the release of VEGF into conditioned media. Further analysis of gelatinase activity of these media samples showed a dose dependent and time dependent decrease in the amount of matrix-metalloproteinase-2 activity (FIG. 12C), and thus the quantity of mature matrix-metalloproteinase-2 that was released into the media. The data suggests that after 10 minutes of treatment, there was a dose dependent decrease in the levels of mature matrix-metalloproteinase-2 released into the media. This decrease was more prominent in samples collected at 30 minutes and 24 hours after treatment.

Glioma cells express several matrix-metalloproteinases including matrix-metalloproteinase-1, matrix-metalloproteinase-3 and matrix-metalloproteinase-9. Of these, matrix-metalloproteinase-2 and matrix-metalloproteinase-9 are specifically upregulated in gliomas. Therefore it was investigated whether chlorotoxin could also interact with pure matrix-metalloproteinase-1, matrix-metalloproteinase-3 or matrix-metalloproteinase-9. His-chlorotoxin only interacted with pure recombinant matrix-metalloproteinase-2, and no detectable binding was observed with matrix-metalloproteinase-1, matrix-metalloproteinase-3 or matrix-metalloproteinase-9 in an overlay assay (FIG. 12D). These findings indicate that chlorotoxin is a specific matrix-metalloproteinase-2 inhibitor that significantly inhibits the release of mature matrix-metalloproteinase-2 from glioma cells.

### EXAMPLE 9

Chlorotoxin Induces Internalization of the Cltx-Complex

The inhibition of matrix-metalloproteinase-2 release following treatment with chlorotoxin is a prolonged effect relative to the rapid inhibition of enzymatic activity by chlorotoxin, suggesting that a cellular mechanism such as endocytosis of matrix-metalloproteinase-2 may be involved in the loss of secreted matrix-metalloproteinase-2. Receptor mediated endocytosis has been found to occur either by a clathrin mediated pathway or by caveolae, Given that other toxins and integrins were internalized via caveolae, whether chlorotoxin causes internalization of cell surface matrix-metalloproteinase-2 and whether this may involve caveolae were examined.

To this end, D54-MG glioma cells were treated for 30 min with 500 μ M chlorotoxin at 37° C. Cells were then fixed and stained under either unpermeabilized or permeabilized conditions. The former only detects cell surface matrix-metalloproteinase-2, while the latter reveals the distribution of both surface and intracellular matrix-metalloproteinase-2.

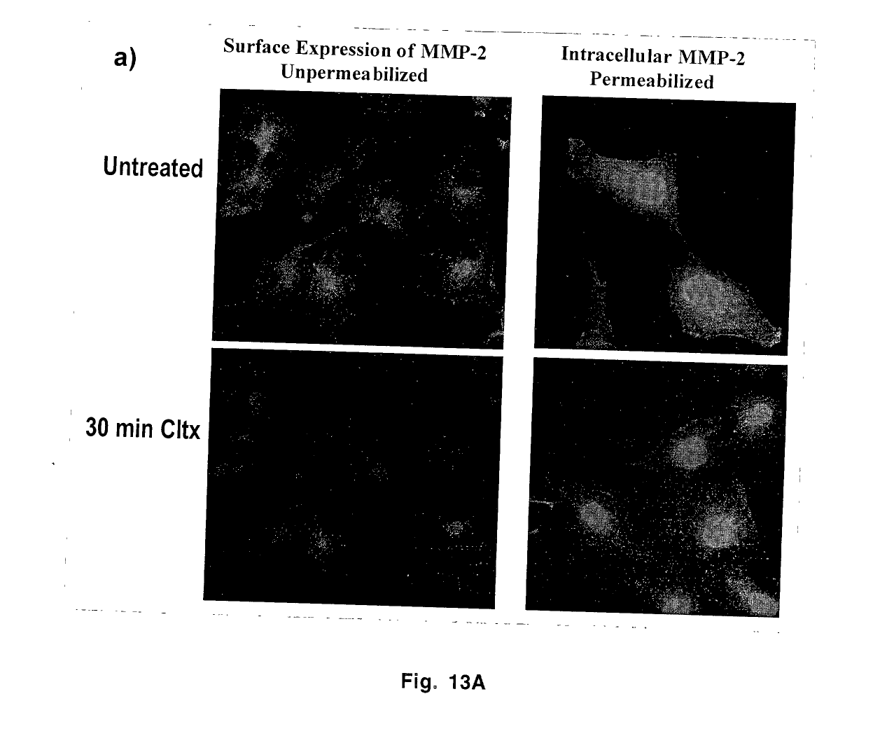

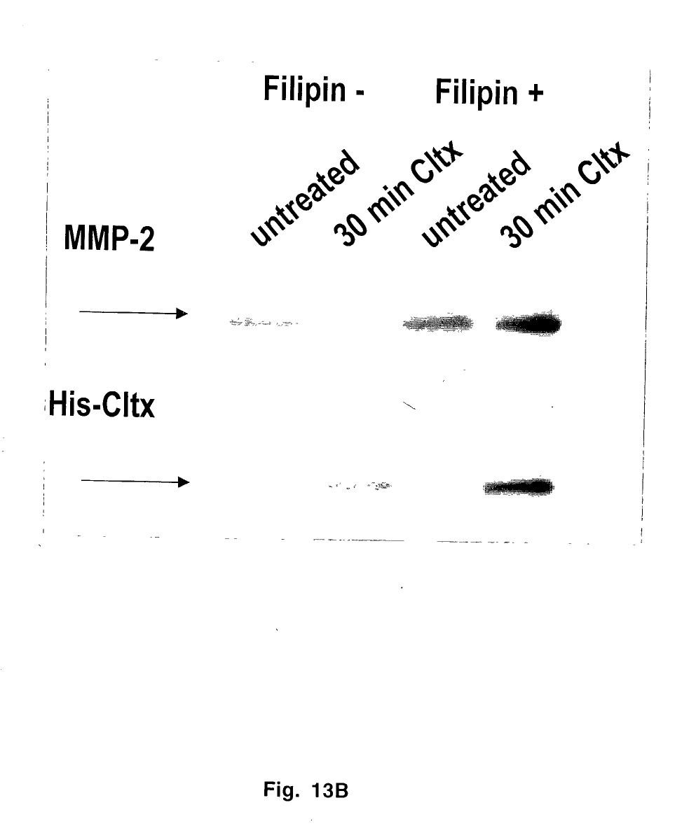

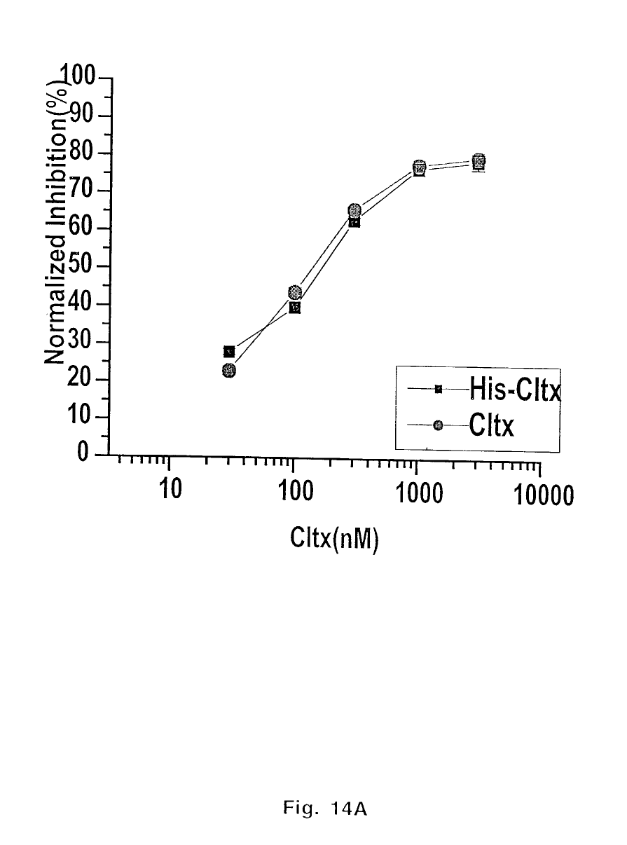

Prominent expression of matrix-metalloproteinase-2 was observed in untreated glioma cells both at the cell surface and intracellularly (FIG. 13A). However, following a 30 minute treatment with chlorotoxin, surface staining for matrix-metalloproteinase-2 was essentially absent whereas intracellular staining remained. These data suggest that His-chlorotoxin reduces surface expression of matrix-metalloproteinase-2 and may do so by inducing increased internalization of matrix-metalloproteinase-2.

To assess whether this observed matrix-metalloproteinase-2 internalization by chlorotoxin is via caveolae, cell surface biotinylation experiments were performed in the presence and absence of Filipin, a sterol binding drug known to disrupt caveolae. Here, cell surface membrane proteins were labeled with biotin and isolated using Avidin beads from either untreated control cells or cells treated with chlorotoxin for 30 min at 37° C. in the presence or absence of Filipin. In the presence of Filipin, a significantly larger fraction of His-Cltx as well as matrix-metalloproteinase-2 remained on the cell surface, whereas in the absence of Filipin, significant reduction in membrane associated His-Cltx and matrix-metalloproteinase-2 was observed (FIG. 13B). These data suggest that a Filipin-sensitive mechanism, possibly caveolae, is involved in the internalization of matrix-metalloproteinase-2 following binding of chlorotoxin.

### EXAMPLE 10

Chlorotoxin Blocks Invasion of Glioma Cells

Invasion through extracellular matrix is a crucial step in tumor metastasis. As chlorotoxin blocks directional migration, the anti-invasive properties of chlorotoxin was investigated utilizing a matrigel invasion assay. Matrigel matrix is a reconstituted basement membrane isolated from Englebreth-Holm-Swarm (EHS) mouse sarcoma, a tumor rich in extracellular matrix proteins. Matrigel-invasion chamber consisted of falcon cell culture inserts of 8 μm pore-size with a uniform layer of matrigel matrix that occludes the membrane pores. After rehydration of the inserts, glioma cells were plated at a density of 5×10⁴ in chambers that were coated or not coated with vitronectin and were treated with 500 nM chlorotoxin. Cells which remained in the upper chamber were scrubbed off the inserts and the invaded cells were fixed and stained with crystal violet.

Based on the above data, one would expect that chlorotoxin binding to matrix-metalloproteinase-2 should reduce glioma cell invasion either by the inhibition of its enzymatic activity or by decreasing surface expression and/or release of matrix-metalloproteinase-2. The inhibitory properties of chlorotoxini on glioma matrigel invasion were examined by comparing the effect of His-chlorotoxin to commercially available peptide.

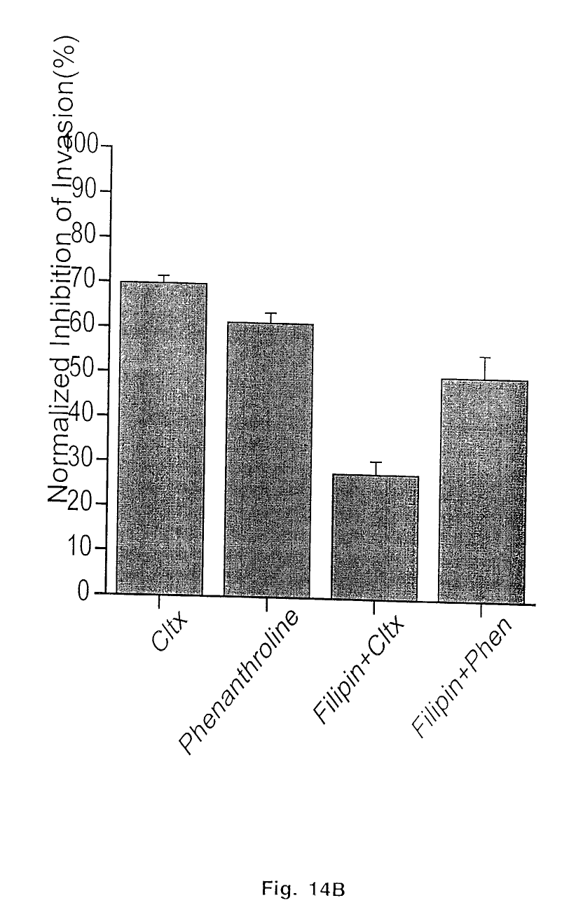

Both peptides inhibited invasion in a concentration-dependent manner with an IC-50 of about 200 nM, a value essentially identical to IC-50 for the inhibition of matrix-metalloproteinase-2 activity by chlorotoxin (see FIG. 11B). The maximal inhibition obtained with chlorotoxin was between 70-80% as compared to untreated control cells (FIG. 14A). Interestingly, addition of 1-10 phenanthroline yielded essentially identical inhibition of invasion as did chlorotoxin (FIG. 14B). However, when the effects of either chlorotoxin or 1-10 phenanthroline were examined in the presence of Filipin, the effect of chlorotoxin was reduced by over 50%, indicating that a significant component of the chlorotoxin effect on matrigel invasion presumably involves the endocytosis of matrix-metalloproteinase-2 via caveolae. Consistent with this concept, it was found that inhibition of glioma invasion by 1-10 phenanthroline, which specifically inhibits the enzymatic activity of matrix-metalloproteinase-2, was only minimally affected by treatment with Filipin. Taken together, these findings suggest a novel mechanism of action for this scorpion toxin, wherein chlorotoxin regulates invasion by modulating the surface expression of enzymatically active matrix-metalloproteinase-2.

The present invention has significant therapeutic implications. The anti-invasive effects of chlorotoxin on glioma cells suggest that this drug may be highly useful in the treatment of malignant gliomas. Indeed, chlorotoxin has passed preclinical safety studies and has recently won FDA approval for use in a Phase I/II clinical trial. Several embryologically related tumors, including melanomas, have also been shown to express matrix-metalloproteinase-2 and to bind chlorotoxin. Clinical use of chlorotoxin may thus be expanded to include these tumors as well. Importantly, however, being a specific matrix-metalloproteinase-2 inhibitor, chlorotoxin may have even broader utility. Matrix-metalloproteinase-2 is involved in a range of diseases that involve tissue remodeling in disease progression. Several chemical inhibitors of MMP-2 are in various stages of clinical testing but most have failed due to toxicity or lack of specificity. Chlorotoxin would be a safer and more specific drug, worthy of further exploration in this context.

The following references are cited herein:

Armstrong et al., Can. J. Cardiol., 10:214-220 (1994).

Bendeck et al., Circulation Research, 75:539-545 (1994).

Benelli et al., Oncology Research, 6:251-257 (1994).

Brown et al., Cancer Res., 50:6184-6191 (1990).

DeClerck et al., Cancer Res., 52:701-708 (1992).

Deryugina et al., J. Cell Sci. 110:2473-2482 (1997).

Ellerbroek and Stack, BioEssays 21:940-949 (1999).

Friedberg et al., Cancer 82:923-930 (1998).

Grams et al., Eur. J. Biochem., 228:830-841 (1995).

Hidalgo et al., Adv. Intern. Med 47:159-190 (2001).

Kahari and Saarialho Kere, Ann. Med 31:34-45 (1999).

Kitamura et al., Kidney Int., 45:1580-1586 (1994).

Lee et al., Am. J. Cardiol., 72:672-676 (1993).

Marti et al., Biochem. J., 291:441-446 (1993).

Monsky et al., Cancer Res., 53:3159-3164 (1993).

Oh et al., J. Neurosci. 19:8464-8475 (1999).

Pauly et al., Circulation Research, 75:41-54 (1994).

Ramanic et al., J. Cell Biology, 125:1165-1178 (1994).

Reddy et al., Clin. Res., 41:660A (1993).

Sabbah et al.,Am. J. Physiol., 263:H266-270 (1992).

Sawaya et al., Clin. Exp. Metastasis 14:35-42 (1996).

Soroceanu et al., J. Neurosci. 19:5942-5954 (1999).

Strongin et al., Biol. Chem., 268:14033-14039 (1993).

Taraboletti et al., Journal of the National Cancer Institute, 87:293 (1995).

Turck et al., J. Biol. Chem., 271:15074-15083 (1996).

Vincenti et al., Arthritis Rheum., 8: 1115-1126 (1994).

Vine and Powell, Clin. Sci., 81:233-239 (1991).

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

One skilled in the art will readily appreciate that the present invention is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A method of treating an individual having a pathophysiological condition that involves the activity of matrix metalloproteinase-2 (MMP-2)/pro-MMP2 system, comprising the step of:
administering to said individual a pharmaceutical composition comprising a pharmaceutically effective dose of chlorotoxin and a pharmaceutically acceptable carrier.

2. The method of claim 1, wherein said chlorotoxin is selected from the group consisting of native chlorotoxin, synthetic chlorotoxin and recombinant chlorotoxin.

3. The method of claim 1, wherein said chlorotoxin is administered in a dose of from about 0.01 mg/kg of body weight of the individual to about 100 mg/kg of body weight of the individual.

4. The method of claim 3, wherein said chlorotoxin is administered in a route selected from the group consisting of intravenous, intramuscular, intracranial and intrathecal administration.

5. The method of claim 1, wherein said pathophysiological condition that involves the activity of matrix metalloproteinase-2/pro-MMP2 system is cancer.

6. The method of claim 5, wherein said cancer is selected from the group consisting of melanoma, breast carcinoma, glioma, pancreatic cancer, small lung cell carcinoma, ovarian carcinoma, colorectal cancer and urothelial cancer.

7. The method of claim 1, wherein said pathophysiological condition that involves the activity of matrix metalloproteinase-2/pro-MMP2 system is metastasis of tumor cells.

8. The method of claim 1, wherein said pathophysiological condition that involves the activity of matrix metalloproteinase-2/pro-MMP2 system is an autoimmune or inflammatory disorders that is dependent on the tissue invasion of leukocytes or other activated migrating cells.

9. The method of claim 8, wherein said pathophysiological condition is selected from the group consisting of arthritis, osteoporosis, multiple sclerosis and renal disease.

10. The method of claim 1, wherein said pathophysiological condition that involves the activity of matrix metalloproteinase-2/pro-MMP2 system is selected from the group consisting of treatment of atherosclerotic plaque rupture, aortic aneurism, heart failure, restenosis, periodontal disease, corneal ulceration, treatment of burns, decubital ulcers, wound repair, inflammation and pain.

11. The method of claim 1, wherein said pathophysiological condition that involves the activity of matrix metalloproteinase-2/pro-MMP2 system is a neurodegenerative disorder.

12. The method of claim 11, wherein said neurodegenerative disorder is selected from the group consisting of stroke, head trauma, spinal cord injury, Alzheimer's disease, amyotrophic lateral sclerosis, cerebral amyloid angiopathy, AIDS, Parkinson's disease, Huntington's disease, prion diseases, myasthenia gravis and Duchenne's muscular dystrophy.

13. A method of inhibiting neoplastic cells or metastasis of neoplastic cells, comprising the step of:
administering to said cells a pharmaceutical composition comprising a pharmaceutically effective dose of chlorotoxin and a pharmaceutically acceptable carrier.

14. The method of claim 13, wherein said chlorotoxin is selected from the group consisting of native chlorotoxin, synthetic chlorotoxin and recombinant chlorotoxin.

15. The method of claim 13, wherein said chlorotoxin is administered in a dose of from about 0.01 mg/kg of body weight of the individual to about 100 mg/kg of body weight of the individual.

16. The method of claim 13, wherein said neoplastic cell is selected from the group consisting of melanoma cel, breast carcinoma cells, glioma cells, pancreatic cancer cells, small lung cell carcinoma cells, ovarian carcinoma cells, colorectal cancer cells and urothelial cancer cells.

17. A method of treating an autoimmune or inflammatory disorder in an individual in need of such treatment, wherein said disorder is dependent on the tissue invasion of leukocytes or other activated migrating cells, comprising the step of:
administering to said individual a pharmaceutical composition comprising a pharmaceutically effective dose of chlorotoxin and a pharmaceutically acceptable carrier.

18. The method of claim 17, wherein said chlorotoxin is selected from the group consisting of native chlorotoxin, synthetic chlorotoxin and recombinant chlorotoxin.

19. The method of claim 17, wherein said chlorotoxiin is administered in a dose of from about 0.01 mg/kg of body weight of the individual to about 100 mg/kg of body weight of the individual.

20. The method of claim 17, wherein said autoimmune or inflammatory disorder is selected from the group consisting of arthritis, osteoporosis, multiple sclerosis and renal disease.

21. A method of treating pathophysiological condition involves the activity of matrix metalloproteinase-2/pro-MMP2 system in an individual in need of such treatment, wherein said condition is selected from the group consisting of treatment of atherosclerotic plaque rupture, aortic aneurism, heart failure, restenosis, periodontal disease, corneal ulceration, treatment of burns, decubital ulcers, wound repair, inflammation and pain, comprising the step of:
administering to said individual a pharmaceutical composition comprising a pharmaceutically effective dose of chlorotoxin and a pharmaceutically acceptable carrier.

22. The method of claim 21, wherein said chlorotoxin is selected from the group consisting of native chlorotoxin, synthetic chlorotoxin and recombinant chlorotoxin.

23. The method of claim 21, wherein said chlorotoxin is administered in a dose of from about 0.01 mg/kg of body weight of the individual to about 100 mg/kg of body weight of the individual.

24. A method of treating a neurodegenerative disorder involves the activity of matrix metalloproteinase-2/pro-MMP2 system in an individual in need of such treatment, comprising the step of:
administering to said individual a pharmaceutical composition comprising a pharmaceutically effective dose of chlorotoxin and a pharmaceutically acceptable carrier.

25. The method of claim 24, wherein said chlorotoxin is selected from the group consisting of native chlorotoxin, synthetic chlorotoxin and recombinant chlorotoxin.

26. The method of claim 24, wherein said chlorotoxin is administered in a dose of from about 0.01 mg/kg of body weight of the individual to about 100 mg/kg of body weight of the individual.

27. The method of claim 24, wherein said neurodegenerative disorder is selected from the group consisting of stroke, head trauma, spinal cord injury, Alzheimer's disease, amyotrophic lateral sclerosis, cerebral amyloid angiopathy, AIDS, Parkinson's disease, Huntington's disease, prion diseases, myasthenia gravis and Duchenne's muscular dystrophy.
